(19) United States
(12) Patent Application Publication
(10) Pub. No.: US 2017/0079763 A1
(43) Pub. Date: Mar. 23, 2017

(54) MEDICAL DEVICE

(71) Applicant: OLYMPUS CORPORATION, Tokyo (JP)

(72) Inventors: Jun Honda, Tokyo (JP); Takahiro Ono, Tokyo (JP); Masaki Hayashi, Tokyo (JP)

(73) Assignee: OLYMPUS CORPORATION, Tokyo (JP)

(21) Appl. No.: 15/370,560

(22) Filed: Dec. 6, 2016

Related U.S. Application Data
(63) Continuation of application No. PCT/JP2015/054561, filed on Feb. 19, 2015.

(30) Foreign Application Priority Data
Jun. 23, 2014 (JP) 2014-128215

(51) Int. Cl. A61F 2/00; A61F 7/00; A61F 7/12

(52) U.S. Cl. CPC A61F 2/0036; A61F 7/12; A61F 7/0085; A61F 2210/0085; A61F 2007/126

(57) ABSTRACT

A medical device including: a needle that has an inner hole formed so as to penetrate the needle from a distal end to a base end in the longitudinal direction; a medical-fluid supplying section that is connected to the base end of the needle and that supplies, to the inner hole, a medical fluid that hardens when warmed or cooled; a temperature changing member that is provided at the distal end of the needle or in the vicinity of the distal end thereof and that can be warmed or cooled when supplied with energy; and a temperature adjusting section that warms or cools the temperature changing member by supplying the energy to the temperature changing member.

### CROSS-REFERENCE TO RELATED APPLICATIONS

This is a continuation of International Application PCT/JP2015/054561, with an international filing date of Feb. 19, 2015, which is hereby incorporated by reference herein in its entirety. This application claims the benefit of Japanese Patent Application No. 2014-128215, filed on Jun. 23, 2014, the content of which is incorporated herein by reference.

### TECHNICAL FIELD

The present invention relates to a medical device.

### BACKGROUND ART

A medical device for treating women's urinary incontinence is conventionally known (for example, see PTL 1). This medical device is provided with an elongated shaft and a needle that can be protruded from a distal end of the shaft. From the needle, with which the urethral wall is punctured, a medical fluid is injected between a mucosal layer and a submucosal layer to locally raise the urethral wall, thus narrowing part of the urethra, thereby making it possible to remedy urinary incontinence.

### CITATION LIST

### Patent Literature

{PTL 1} Japanese Translation of PCT International Application, Publication No. 2006-501039

### SUMMARY OF INVENTION

### Technical Problem

In usual treatment for urinary incontinence, a medical fluid is injected into at least three locations in order to evenly raise the urethral wall along the entire circumference. At this time, if the needle is pulled out from the urethral wall immediately after the medical fluid is injected, the medical fluid may leak out of the urethral wall via an opening in the urethral wall.

The present invention is a medical device capable of moving on to the injection operation at the next location immediately after injection of the medical fluid into the wall of the body cavity, thereby making it possible to reduce the burden associated with the operation and to achieve a reduction in the treatment time.

### Solution to Problem

An one aspect of the present invention provides a medical device including: a needle that has an inner hole formed so as to penetrate the needle from a distal end to a base end in the longitudinal direction; a medical fluid supplying section that is connected to the base end of the needle and that supplies, to the inner hole, a medical fluid that hardens when warmed or cooled; a temperature changing member that is provided at the distal end of the needle or in the vicinity of the distal end thereof and that can be warmed or cooled when supplied with energy; and a temperature adjusting section that warms or cools the temperature changing member by supplying the energy to the temperature changing member.

In the above-described aspect, the temperature adjusting section may warm the temperature changing member to 30° C. or higher and 50° C. or lower. A collagen solution can be preferably used as a medical fluid that hardens when warmed to 30° C. or higher and 50° C. or lower.

In the above-described aspect, the temperature adjusting section may cool the temperature changing member to 10° C. or higher and 30° C. or lower.

In the above-described aspect, the temperature adjusting section may be provided with: an energy source that is provided at a location close to the base end of the needle and that outputs the energy; and an energy transfer member that extends in the longitudinal direction of the needle in parallel with the needle to connect the energy source with the temperature changing member and that transfers the energy output from the energy source to the temperature changing member; and a heat blocking section that intervenes between the needle and the energy transfer member to block the heat between the energy transfer member and the needle may be further provided.

In the above-described aspect, it may be possible to further include a cylindrical sheath that has a through-hole penetrating the sheath from a distal-end surface to a base-end surface and accommodating the needle so as to allow the needle to move in the longitudinal direction.

In the above-described aspect, the sheath may be provided with a fixing section that fixes a distal end of the sheath to the wall of a body cavity.

In the above-described aspect, the fixing section may be a plurality of projections that project from the distal-end surface of the sheath.

In the above-described aspect, the fixing section may be a pair of grasping members that are provided at the distal end of the sheath and that grasp the wall of the body cavity.

In the above-described aspect, the temperature changing member may be provided on the distal-end surface of the sheath.

### BRIEF DESCRIPTION OF DRAWINGS

FIG. 1 is a view showing the overall configuration of a medical device according to one embodiment of the present invention.

FIG. 2A is a view for explaining how to use the medical device shown in FIG. 1 and showing a state in which a distal-end surface of a sheath is brought into close contact with the surface of a urethral wall.

FIG. 2B is a view for explaining how to use the medical device shown in FIG. 1 and showing a state in which the urethral wall is punctured with a needle, and a medical fluid is injected.

FIG. 2C is a view for explaining how to use the medical device shown in FIG. 1 and showing a state in which the needle is pulled out from the urethral wall.

FIG. 3 is a perspective view of a distal-end portion showing a modification of the medical device shown in FIG. 1.

FIG. 4 is a side view of a distal-end portion showing another modification of the medical device shown in FIG. 1.

FIG. 5A is a view for explaining how to use the medical device shown in FIG. 4 and showing a state in which the urethral wall is punctured with the needle.

FIG. 5B is a view for explaining how to use the medical device shown in FIG. 4 and showing a state in which a medical fluid is injected.

FIG. 5C is a view for explaining how to use the medical device shown in FIG. 4 and showing a state in which the urethral wall is grasped with a pair of grasping members.

FIG. 5D is a view for explaining how to use the medical device shown in FIG. 4 and showing a state in which the needle is pulled out from the urethral wall.

FIG. 6 is a view of the overall configuration showing still another modification of the medical device shown in FIG. 1.

### DESCRIPTION OF EMBODIMENT

A medical device 1 according to one embodiment of the present invention will be described below with reference to the drawings.

The medical device 1 of this embodiment is an endoscope treatment tool used by being inserted into a treatment-tool channel formed in an elongated insertion portion of an endoscope and is used to inject a medical fluid A inside a wall of the body cavity while observing the inside of the body cavity with the endoscope, to locally raise the wall of the body cavity.

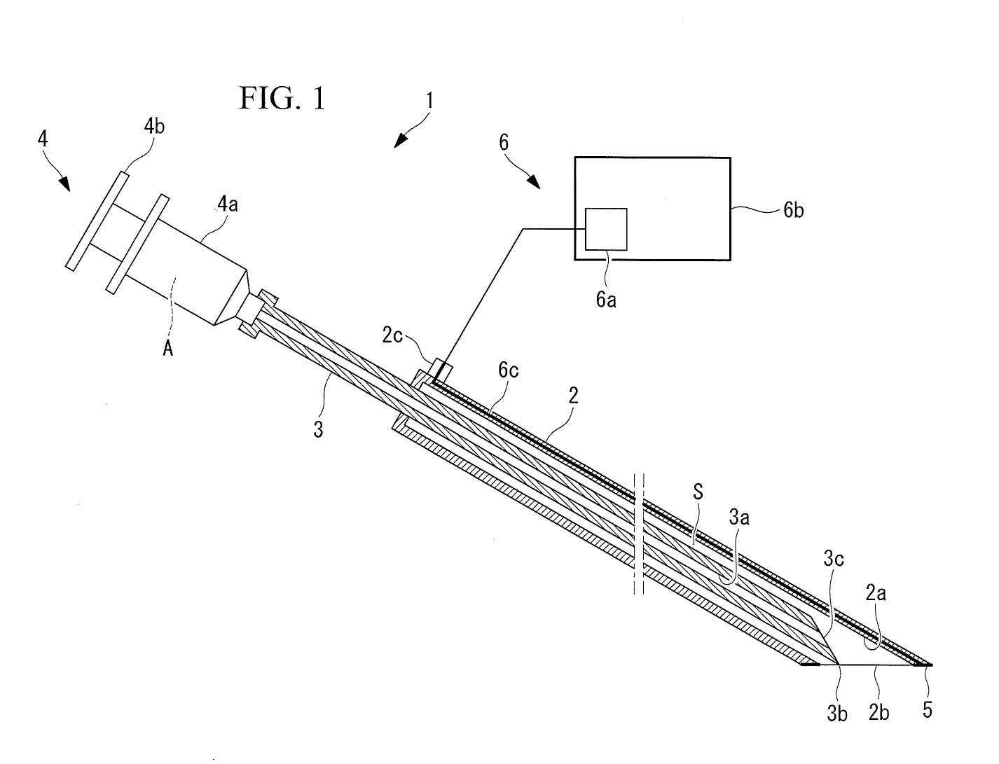

As shown in FIG. 1, the medical device 1 is provided with: a thin sheath 2; a needle 3 that is accommodated in the sheath 2; a medical-fluid supplying section 4 that is attached to a base end of the needle 3; a warming member (temperature changing member) 5 that is provided on a distal-end surface 2b of the sheath 2; and a temperature adjusting section 6 that adjusts the temperature of the warming member 5.

The sheath 2 is an elongated cylindrical member and is provided with: a through-hole 2a that penetrates the sheath 2 in the long-axis direction; and the distal-end surface 2b, which is located at the distal end thereof and is inclined with respect to the long axis.

The needle 3 is an elongated cylindrical member that is thinner than the sheath 2 and is provided with an inner hole 3a that penetrates the needle 3 in the long-axis direction. A distal-end surface of the needle 3 is inclined with respect to the long axis, and a distal end thereof is a sharp apex 3b. The outer-diameter size of the needle 3 is designed to be smaller than the inner-diameter size of the through-hole 2a such that a ring-like gap, i.e., an air space S, is formed between the outer periphery of the needle 3 and the inner wall of the through-hole 2a of the sheath 2.

The medical-fluid supplying section 4 is provided with: a syringe 4a whose discharge port is connected to the base end of the needle 3; and a plunger 4b that is inserted into the syringe 4a. By pushing the plunger 4b, the medical fluid A contained in the syringe 4a can be supplied from the discharge port to the inner hole 3a of the needle 3, and the medical fluid A can be discharged from an opening 3c in the distal-end surface of the needle 3.

The medical fluid A has a solgel transition temperature between room temperature (about 25° C.) and body temperature (about 37° C.), turns into a sol having a low viscosity capable of flowing in the inner hole 3a of the needle 3 at room temperature, and turns into a gel having a high viscosity at body temperature. It is preferred that the medical fluid A contain, as a component, at least one of: collagen, glycosaminoglycan (hyaluronic acid, chondroitin sulfate, heparin, etc.), cellulose, amylose, dextran, trehalose, chitosan, alginic acid, polylactic acid, polyglycolic acid, and albumin; and a derivative thereof; a salt thereof; and a polymer thereof.

The warming member 5 is made of a high-thermal-conductivity metal film that is formed on the distal-end surface of the needle 3 in a ring-like manner so as to surround the entire circumference of the opening 3c of the inner hole 3a.

The temperature adjusting section 6 is provided with: a temperature control device 6b that has a heat source (energy source) 6a, such as a heater, provided at a location close to the base end of the sheath 2; and a heat transfer wire (energy transfer member) 6c that connects the heat source 6a in the temperature control device 6b with the warming member 5.

The heat transfer wire 6c is made of a high-thermal-conductivity metal thin wire and extends from the distal end of the sheath 2 while passing through the inside of the sidewall of the sheath 2 to a plug 2c that is provided at a base-end portion of the sheath 2 and further extends from the plug 2c to the heat source 6a.

The temperature control device 6b is connected to the heat transfer wire 6c via the plug 2c. When the temperature control device 6b activates the heat source 6a to raise the temperature of the heat source 6a, the heat of the heat source 6a is transferred to the heat transfer wire 6c and is further transferred to the warming member 5, thus warming the warming member 5. At this time, the temperature control device 6b adjusts the temperature of the heat source 6a such that the warming member 5 is warmed at 30° C. or higher and 50° C. or lower.

Next, the operation of the thus-configured medical device 1 of this embodiment will be described by taking, as an example, treatment for urinary incontinence in which the medical fluid A is injected between a mucosal layer and a submucosal layer of a urethral wall B.

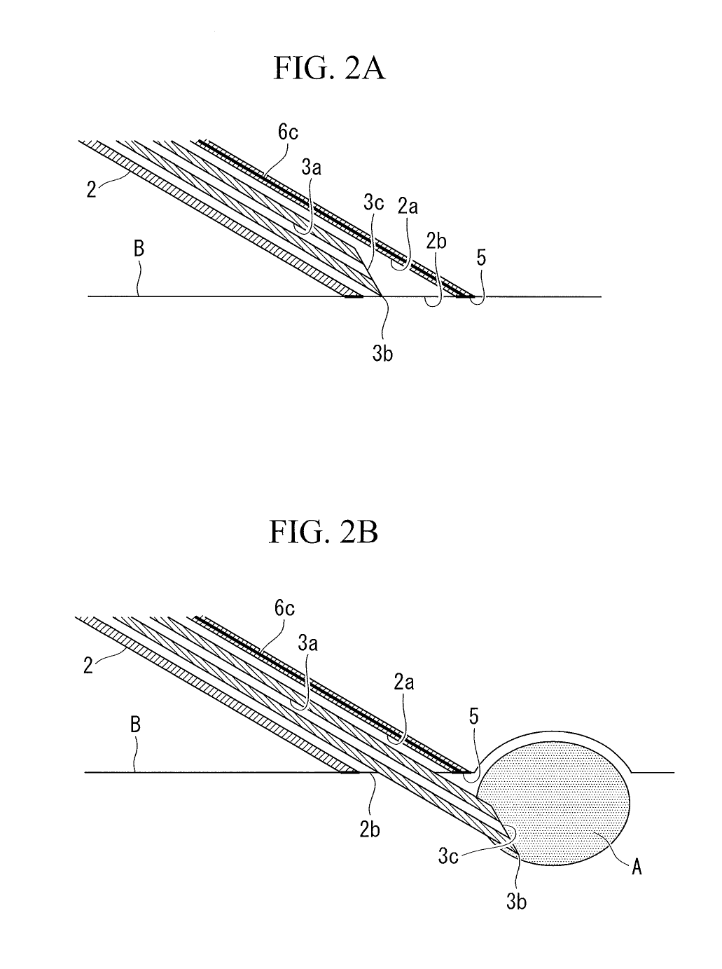

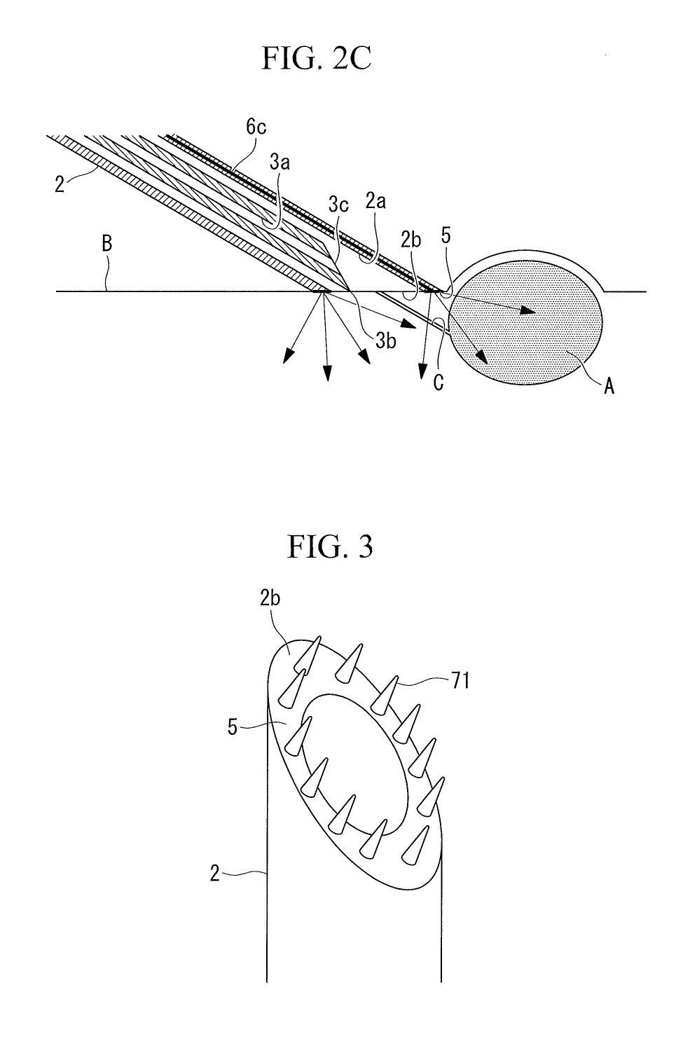

In order to treat urinary incontinence by using the medical device 1 of this embodiment, first, the insertion portion of the endoscope is inserted into the urethra of a patient. Next, the needle 3 and the sheath 2 are inserted into the urethra through the treatment-tool channel of the insertion portion, with the apex 3b of the needle 3 withdrawn into the sheath 2, as shown in FIG. 2A, and the distal-end surface 2b of the sheath 2 is brought into contact with the surface of the urethral wall B. The distal-end surface 2b of the sheath 2 is inclined with respect to the long axis of the sheath 2; thus, in the state shown in FIG. 2B, the sheath 2 is disposed inclined with respect to the surface of the urethral wall B.

Next, while maintaining the position of the sheath 2 with respect to the urethral wall B, the needle 3 is moved with respect to the sheath 2 toward the distal end, thereby making the needle 3 protrude from the distal-end surface 2b of the sheath 2, as shown in FIG. 2B, puncturing the urethral wall B with the apex 3b, and disposing the distal-end surface of the needle 3 between the mucosal layer and the submucosal layer. Next, the plunger 4b of the medical-fluid supplying section 4 is pressed, thereby discharging the medical fluid A in the syringe 4a from the opening 3c via the inner hole 3a of the needle 3. The discharged medical fluid A is accumulated between the mucosal layer and the submucosal layer, thereby locally raising the urethral wall B.

Next, while maintaining the position of the sheath 2 with respect to the urethral wall B, the needle 3 is moved with respect to the sheath 2 toward the base end, and thus, the needle 3 is pulled out from the urethral wall B, as shown in FIG. 2C, to withdraw the apex 3b into the sheath 2. Next, the temperature control device 6b raises the temperature of the heat source 6a to start warming the warming member 5. Accordingly, the medical fluid A located at a contact portion of the urethral wall B with the warming member 5 and in the vicinity of this contact portion is warmed, thereby promoting hardening of the medical fluid A and making the medical fluid A rapidly harden into a gel.

Next, the distal-end surface 2b of the sheath 2 is moved to other treatment locations on the urethral wall B, and the same operation is repeated; thus, three locations in total on the urethral wall B that are about 120° C. away from each other in the circumferential direction are raised such that the urethral wall B is almost-equally raised along the entire circumference. Accordingly, it is possible to treat the urinary incontinence by partially narrowing the urethra.

In this case, according to the medical device 1 of this embodiment, when the medical fluid A injected inside the urethral wall B is warmed by using the warming member 5, the medical fluid A rapidly hardens. After the medical fluid A hardens, even when the needle 3 is pulled out from the urethral wall B, the medical fluid A does not leak from a hole C opened in the urethral wall B. Therefore, a doctor can pull out the needle 3 from the urethral wall B immediately after injecting the medical fluid A, without waiting for a long time, and can move on to the operation of injecting the medical fluid A into another location. Accordingly, there is an advantage in that it is possible to reduce the burden associated with the operation, thus shortening the time required for the treatment.

Furthermore, although the heat transfer wire 6c, which transfers heat from the heat source 6a to the warming member 5, is also warmed, the air space (heat blocking section) S, which intervenes between the inner periphery of the sheath 2 and the outer periphery of the needle 3, functions as a heat barrier, so that the heat transfer from the heat transfer wire 6c to the needle 3 is blocked. Accordingly, there is an advantage in that it is possible to avoid a situation in which the medical fluid A in the inner hole 3a of the needle 3 is warmed by the heat of the heat transfer wire 6c, thus decreasing the viscosity of the medical fluid A.

In this embodiment, as shown in FIG. 3, a plurality of projections (fixing section) 71 that project from the distal-end surface 2b of the sheath 2 may be provided on the distal-end surface 2b. In this case, the metal film forming the warming member 5 may be provided only on a flat portion on the distal-end surface 2b except for the projections 71 or may be provided also on the surfaces of the projections 71.

By doing so, the projections 71 dig into the urethral wall B in a state in which the distal-end surface 2b of the sheath 2 is in close contact with the surface of the urethral wall B, thereby locking the movement of the distal-end surface 2b of the sheath 2 in the direction along the surface of the urethral wall B and temporarily fixing the distal-end surface 2b of the sheath 2 to the urethral wall B. Puncturing of the urethral wall_{—} B with the needle 3 and injection and hardening of the medical fluid A are sequentially performed with the distal-end surface 2b of the sheath 2 fixed to the urethral wall B, thereby allowing the warming member 5 to efficiently warm the medical fluid A and make it harden, with the warming member 5 disposed in the vicinity of the medical fluid A injected inside the urethral wall B.

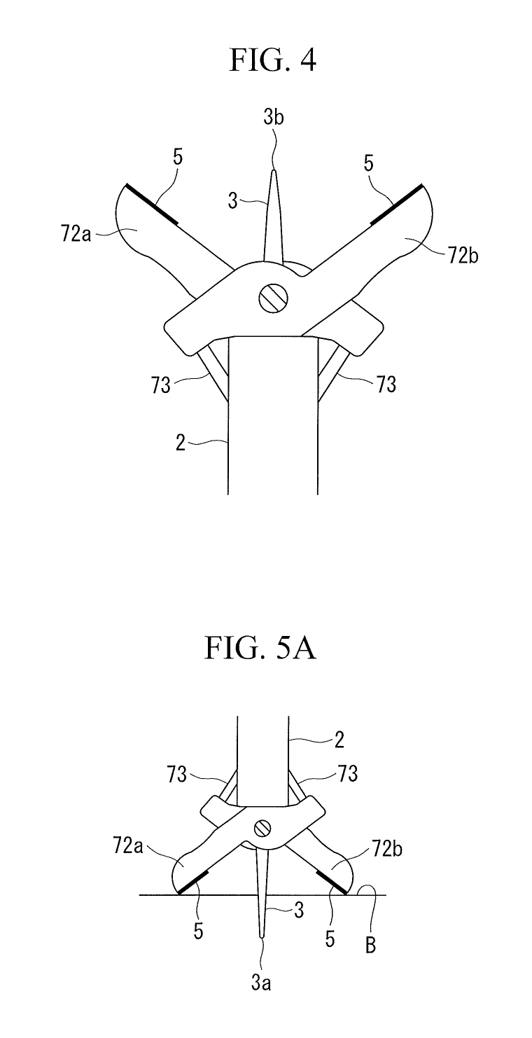

Instead of the projections 71, as shown in FIG. 4, the sheath 2 may be provided with a pair of grasping members 72a and 72b at the distal end thereof, and the distal end of the sheath 2 may be temporarily fixed to the urethral wall B by grasping the urethral wall B with the pair of grasping members 72a and 72b.

The grasping members 72a and 72b are substantially bowl shaped and are coupled so as to be able to open and close, with the curved surfaces thereof directed in opposite directions. In this configuration, warming members 5 are provided at at least distal-end portions on opposing ring-like flat surfaces of the grasping members 72a and 72b.

The needle 3 is provided so as to be able to protrude from between the pair of grasping members 72a and 72b. It is preferred to provide a mechanism that links the open/close movement of the pair of grasping members 72a and 72b with the protrusion/withdrawal movement of the needle 3 such that the needle 3 protrudes toward the distal end when the pair of grasping members 72a and 72b are opened, and the needle 3 withdraws toward the base end when the pair of grasping members 72a and 72b are closed. For example, wires 73 for opening/closing the pair of grasping members 72a and 72b are coupled to the needle 3; when the pair of grasping members 72a and 72b are opened by pushing the wires 73, the needle 3 is also pushed out; and, when the pair of grasping members 72a and 72b are closed by pulling the wires 73, the needle 3 is also pulled.

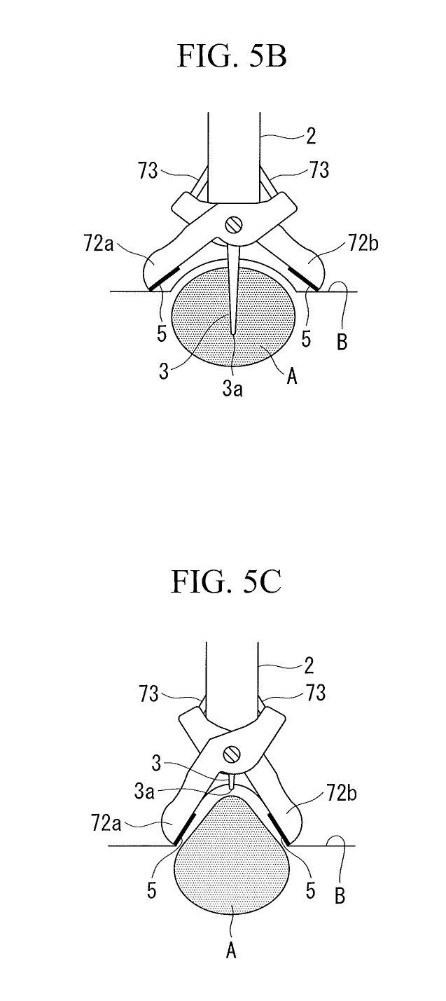

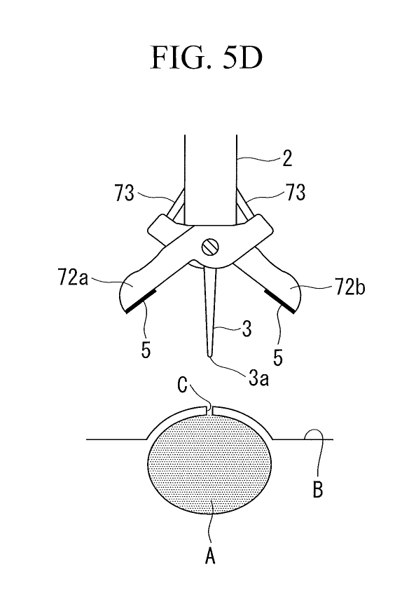

FIGS. 5A to 5D show how to use the medical device 1 provided with the grasping members 72a and 72b. First, the urethral wall B is punctured with the needle 3 with the pair of grasping members 72a and 72b open, as shown in FIG. 5A, and the medical fluid A is injected, as shown in FIG. 5B. Next, the pair of grasping members 72a and 72b are closed to grasp a raised portion of the urethral wall B, as shown in FIG. 5C. At this time, the needle 3 is pulled out from the urethral wall B. Next, while the raised portion is being grasped between the grasping members 72a and 72b, the warming members 5 are warmed to make the medical fluid A harden. Then, as shown in FIG. 5D, the pair of grasping members 72a and 72b are opened.

By doing so, the medical fluid A injected inside the urethral wall B can be efficiently warmed and made to harden by the warming members 5. Note that the shape of the grasping members 72a and 72b is not limited to the bowl shape and can be appropriately changed.

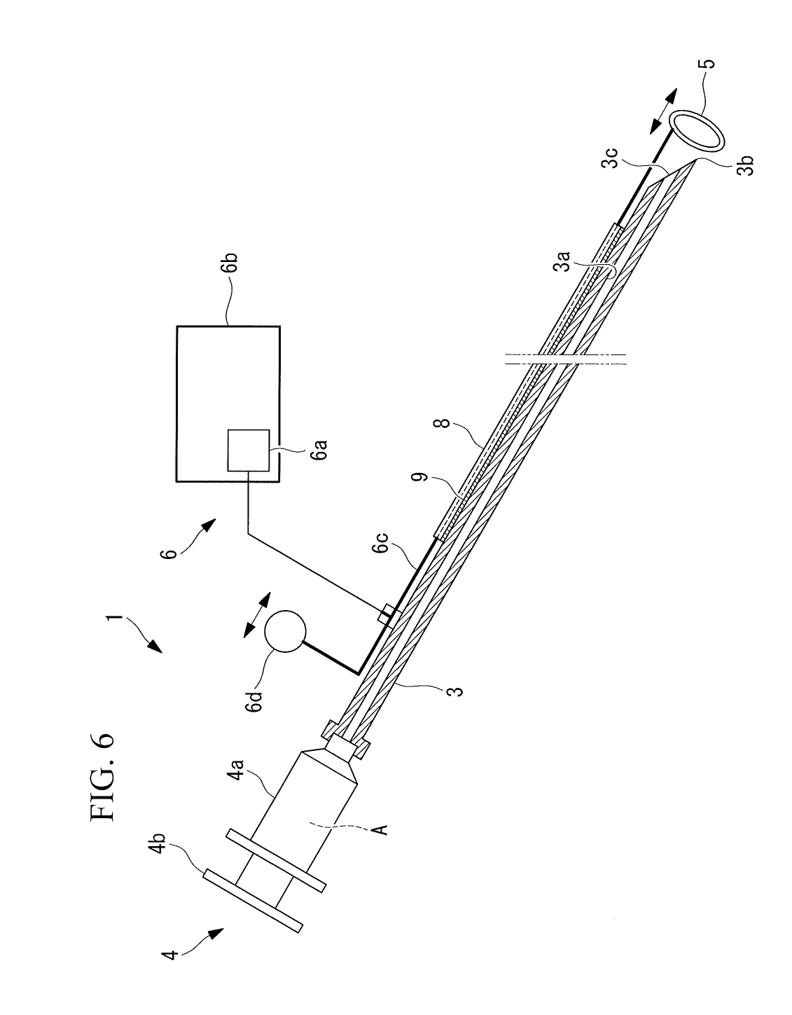

In this embodiment, the sheath 2 is provided; however, the sheath 2 may be omitted. In this case, as shown in FIG. 6, a guide tube 8 that extends in the longitudinal direction of the needle 3 is fixed to the outer periphery of the needle 3, and the heat transfer wire 6c is inserted into the guide tube 8 so as to be movable in the longitudinal direction. The warming member 5 is formed of a ring-like metal thin wire etc. fixed to the distal end of the heat transfer wire 6c, and the warming member 5 can be moved in the front-back directions by manipulating a manipulation unit 6d that is provided at a base-end portion of the heat transfer wire 6c. A heat blocking member (heat blocking section) 9 for blocking heat between the guide tube 8 and the needle 3 is provided between the guide tube 8 and the needle 3.

By doing so, it is possible to achieve a reduction in diameter compared with the configuration having the sheath 2.

In this embodiment, the warming member 5 to be warmed is provided assuming the medical fluid A, which hardens into a gel when warmed; however, instead of this, it is possible to use a medical fluid that hardens into a gel when cooled down to body temperature and to cool a temperature changing member. For example, it is possible to use a cold source instead of the heat source 6a, to use a cooling member that is formed of a metal film, as in the warming member 5, and to transfer the cold of the cold source to the cooling member via the heat transfer wire 6c, thereby cooling the cooling member. In this case, it is preferred that the temperature of the cold source be adjusted so as to cool the cooling member to 10° C. or higher and 30° C. or lower.

In this embodiment, a description has been given of a combination of the heat source 6a and the metal film, as an example combination of the energy source and the temperature changing member; however, the combination of the energy source and the temperature changing member is not limited thereto.

For example, it is also possible to use a current source as an energy source, to use an electric wire as an energy transfer member, and to warm the warming member 5, which is formed of a metal film, when supplied with current. When a current source is used as an energy source, it is possible to use, as a temperature changing member, a heater element that generates heat when supplied with current, such as a resistive element, or an endothermic element that absorbs heat when supplied with current, such as a Peltier element.

Alternatively, it is also possible to use a high-frequency power source as an energy source, to use an ultrasonic transducer as a temperature changing member, and to warm the ultrasonic transducer and the medical fluid A due to ultrasonic vibrations produced by the ultrasonic transducer when supplied with a high-frequency current.

In this embodiment, a description has been given of the medical device 1 used as an endoscope treatment tool; however, the form of the medical device 1 is not limited thereto, and the medical device 1 can be changed to specifications suitable for various treatment methods. For example, the medical device 1 may be used, independently of other medical devices, in laparoscopic surgery or abdominal surgery.

According to the above described embodiment of the medical device, the wall of the body cavity is punctured with the distal end of the needle inserted into the body cavity, and the medical fluid is injected, from the medical fluid supplying section, inside the wall of the body cavity via the inner hole of the needle, thereby making it possible to locally raise the wall of the body cavity. In this case, the energy is supplied from the temperature adjusting section to the temperature changing member, which is located at the distal end of the needle or in the vicinity thereof, to heat or cool the temperature changing member, thereby making it possible to immediately harden the medical fluid accumulated inside the wall of the body cavity. Therefore, it is possible to pull out the needle from the wall of the body cavity immediately after injection of the medical fluid and to move on to the injection operation at the next location, thus making it possible to reduce the burden associated with the operation to achieve a reduction in the treatment time.

Because the temperature adjusting section warms the temperature changing member to 30° C. or higher and 50° C. or lower, the influence on surrounding tissue that would be caused by warming the temperature changing member can be reduced.

Because the temperature adjusting section cools the temperature changing member to 10° C. or higher and 30° C. or lower, the influence on surrounding tissue that would be caused by cooling the temperature changing member can be reduced.

Even when the temperature of the energy transfer member is changed through transfer of the energy, the heat blocking section can prevent the change in the temperature of the energy transfer member from being transferred to the needle. Accordingly, it is possible to prevent the viscosity of the medical fluid from being decreased in the inner hole of the needle due to the transfer of the energy.

By a cylindrical sheath that has a through-hole penetrating the sheath from a distal-end surface to a base-end surface and accommodating the needle so as to allow the needle to move in the longitudinal direction is included, the needle can be moved in the body cavity, with the apex of the needle accommodated in the sheath except when the medical fluid is injected.

By the sheath is provided with a fixing section that fixes a distal end of the sheath to the wall of a body cavity, in a state in which the distal end of the sheath is fixed to the wall of the body cavity with the fixing section, puncturing of the wall of the body cavity with the needle and injection and hardening of the medical fluid are sequentially performed, thereby making it possible to efficiently make the medical fluid harden by using the temperature changing member disposed in the vicinity of the medical fluid inside the wall of the body cavity.

By the fixing section is formed of a plurality of projections that project from the distal-end surface of the sheath, the plurality of projections dig into the wall of the body cavity in a state in which the distal-end surface of the sheath is in close contact with the wall of the body cavity, thereby locking the movement of the distal-end surface of the sheath in a direction along the surface of the wall of the body cavity. Accordingly, it is possible to temporarily fix the distal end of the sheath to the wall of the body cavity without requiring a special operation.

By the fixing section is formed of a pair of grasping members that are provided at the distal end of the sheath and that grasp the wall of the body cavity, it is possible to temporarily fix the distal end of the sheath to the wall of the body cavity through a simple operation in which the wall of the body cavity is grasped with the pair of grasping members.

By the temperature changing member is provided on the distal-end surface of the sheath, the distal-end surface of the sheath is brought into close contact with the wall of the body cavity, thereby making it possible to position the temperature changing member in the vicinity of the medical fluid injected inside the wall of the body cavity.

### REFERENCE SIGNS LIST

1 medical device

2 sheath

2a through-hole

2b distal-end surface

2c plug

3 needle

3a inner hole

3b apex

3c opening

4 medical-fluid supplying section

4a syringe

4b plunger

5 warming member (temperature changing member)

6 temperature adjusting section

6a heat source (energy source)

6b temperature control device

6c heat transfer wire (energy transfer member)

6d manipulation unit

71 projection (fixing section)

72a, 72b grasping member (fixing section)

8 guide tube

9 heat blocking member (heat blocking section)

A medical fluid

B urethral wall

S air space (heat blocking section)

1. A medical device comprising:
a needle that has an inner hole formed so as to penetrate the needle from a distal end to a base end in the longitudinal direction;
a medical fluid supplying section that is connected to the base end of the needle and that supplies, to the inner hole, a medical fluid that hardens when warmed or cooled;
a temperature changing member that is provided at the distal end of the needle or in the vicinity of the distal end thereof and that can be warmed or cooled when supplied with energy; and
a temperature adjusting section that warms or cools the temperature changing member by supplying the energy to the temperature changing member.

2. A medical device according to claim 1, wherein the temperature adjusting section warms the temperature changing member to 30° C. or higher and 50° C. or lower.

3. A medical device according to claim 2, wherein the medical fluid is a collagen solution.

4. A medical device according to claim 1, wherein the temperature adjusting section cools the temperature changing member to 10° C. or higher and 30° C. or lower.

5. A medical device according to claim 1,
wherein the temperature adjusting section is provided with: an energy source that is provided at a location close to the base end of the needle and that outputs the energy; and an energy transfer member that extends in the longitudinal direction of the needle in parallel with the needle to connect the energy source with the temperature changing member and that transfers the energy output from the energy source to the temperature changing member; and
a heat blocking section that intervenes between the needle and the energy transfer member to block the heat between the energy transfer member and the needle is further provided.

6. A medical device according to claim 1, further comprising a cylindrical sheath that has a through-hole penetrating the sheath from a distal-end surface to a base-end surface and accommodating the needle so as to allow the needle to move in the longitudinal direction.

7. A medical device according to claim 6, wherein the sheath is provided with a fixing section that fixes a distal end of the sheath to the wall of a body cavity.

8. A medical device according to claim 7, wherein the fixing section is a plurality of projections that project from the distal-end surface of the sheath.

9. A medical device according to claim 7, wherein the fixing section is a pair of grasping members that are provided at the distal end of the sheath and that grasp the wall of the body cavity.

10. A medical device according to claim 6, wherein the temperature changing member is provided on the distal-end surface of the sheath.
